(19) United States
(12) Patent Application Publication
(10) Pub. No.: US 2026/0137193 A1
(43) Pub. Date: May 21, 2026

(54) MULTI-MODAL THERAPEUTIC CAP ASSEMBLY FOR COSMETIC CONTAINERS

(71) Applicant: SHENZHEN NUON MEDICAL EQUIPMENT CO., LTD., Shenzhen (CN)

(72) Inventors: ALAIN DIJKSTRA, Amstelveen (NL); LI XIANG, Shenzhen (CN); Tang Yuanzhou, Shenzhen (CN); Zhou Zhiliang, Shenzhen (CN); Tu Mingjun, Shenzhen (CN); Huang Jianlong, Shenzhen (CN)

(73) Assignee: SHENZHEN NUON MEDICAL EQUIPMENT CO., LTD., Shenzhen (CN)

(21) Appl. No.: 19/383,589

(22) Filed: Nov. 8, 2025

(30) Foreign Application Priority Data
Nov. 15, 2024 (CN) 2024227935596
Dec. 27, 2024 (CN) 2024232896572
Sep. 8, 2025 (CN) 2025219184585

(51) Int. Cl. A45D 34/04; A45D 34/00; A61N 5/06

(52) U.S. Cl. CPC A45D 34/04; A61N 5/0616; A45D 2034/002; A45D 2200/20; A61N 2005/0632; A61N 2005/0651

(57) ABSTRACT

The present invention relates to a cosmetic container integrated with a multi-modal therapeutic assembly capable of performing phototherapy, thermal therapy, and microcurrent stimulation. The assembly comprises a container body, a connecting cover, and a multi-modal therapeutic device detachably mounted on the connecting cover. The connecting cover includes a covering surface for sealing the container and a receiving groove for storing the therapy device. The multi-modal therapeutic device includes a housing, light therapy lamps, a temperature control element, and microcurrent electrodes, all controlled by an internal circuit board and switch assembly. Magnetic elements positioned between the cover and device provide stable, detachable coupling. The multi-modal therapeutic device is rechargeable through integrated charging electrodes and designed with a finger ring for ease of use. The compact system enables hygienic storage, convenient portability, and enhanced skincare through combined cosmetic and therapeutic functions.

### TECHNICAL FIELD

The present invention relates to the technical field of skincare and cosmetic treatment devices, and more particularly to a cosmetic container integrated with a multifunctional stimulation device capable of providing phototherapy, thermal therapy, vibrational therapy, ultrasonic wave therapy, and microcurrent therapy. The invention specifically pertains to a connecting cover that magnetically couples with a stimulation device, allowing seamless combination of cosmetic product storage and therapeutic skincare treatment within a single compact and portable assembly.

### BACKGROUND

Traditionally, skincare and therapeutic treatments have been performed using separate products and devices. Skincare formulations such as creams, serums, and lotions are applied manually, while therapeutic effects such as light therapy, heating therapy, cooling therapy, or microcurrent stimulation require independent handheld or plug-in devices. These conventional methods not only make the skincare routine cumbersome but also increase the likelihood of misplacing small therapy instruments. Moreover, when these devices are stored separately from cosmetic products, they are often exposed to dust, contamination, or mechanical damage, reducing hygiene and overall user convenience.

Existing skincare containers serve only as storage units and lack any integrated therapeutic capability. Similarly, conventional therapy devices are often bulky, require external power connections, and are inconvenient to carry while traveling. As a result, users find it difficult to combine skincare application with simultaneous treatment, leading to poor absorption of active ingredients and limited treatment efficiency.

The present invention addresses these shortcomings by providing a cosmetic container integrated with a detachable multifunctional therapy device. The device combines phototherapy, thermal regulation, and microcurrent stimulation within a compact housing that attaches magnetically to the container cover. This configuration allows users to perform professional-grade skincare treatments immediately after applying cosmetic formulations, promoting deeper absorption and improved results.

The invention eliminates the need for separate tools, reduces the risk of loss or contamination, and ensures hygienic storage of the therapy surface within the recessed groove of the cover. Additionally, the magnetic coupling, rechargeable design, and ergonomic finger ring make the system portable, easy to handle, and aesthetically refined. By integrating skincare storage and treatment in one compact unit, the invention fills the gap between cosmetic application and therapeutic enhancement, offering a modern, efficient, and user-friendly skincare solution.

### OBJECTS OF THE INVENTION

Some of the objects of the invention are as follows:

An object of the present invention is to provide a cosmetic container integrated with a detachable stimulation device, enabling users to conveniently perform light, thermal, and microcurrent therapy directly in conjunction with cosmetic application.

Another object of the present invention is to provide an alternate configuration of the cosmetic container comprising a connecting cover and a stimulation device as a therapy cover, wherein the stimulation device is magnetically attachable to the cover body, thereby enabling compact storage and preventing misplacement of the therapy device.

Another object of the present invention is to provide a multi-functional stimulation device that delivers phototherapy, temperature regulation, and microcurrent therapy in a single portable structure for enhanced skincare efficacy.

Another object of the present invention is to provide a connecting cover designed to seal a cosmetic container, so that the stimulation device can function both as a lid and a therapeutic applicator, improving portability and hygiene.

Another object of the present invention is to provide a magnetically coupled connection system between the therapy device and the connecting cover, enabling secure attachment, easy detachment, and stable alignment during storage and use.

Another object of the present invention is to provide a light-transmissive therapy surface integrated with electrodes and a thermally conductive plate, allowing combined light, heat, and microcurrent treatments to be applied to the user's skin.

Another object of the present invention is to provide a compact and ergonomic structure having a finger-ring and switch assembly for convenient handling, activation, and control of the therapy functions.

Another object of the present invention is to provide a stimulation device with integrated battery and charging electrodes configured for magnetic charging, thereby enabling cordless operation and easy recharging without disassembly.

Another object of the present invention is to provide a skincare kit that integrates the container body, connecting cover, and stimulation device into a unified system, thereby enhancing user convenience, portability, and storage efficiency.

Another object of the present invention is to provide a reusable and hygienic therapy solution wherein the therapy surface is stored within a recessed groove of the cover body when not in use, preventing contamination and maintaining cleanliness.

Another object of the present invention is to offer an aesthetically pleasing and functional cosmetic assembly that combines therapeutic treatment with skincare product storage, providing a modern, efficient, and user-friendly skincare experience.

### SUMMARY OF THE INVENTION

According to a first aspect of the present invention, a multi-modal therapeutic cap device for promoting absorption of skincare compositions is provided. The multi-modal therapeutic cap device comprises: a housing defining a therapy surface; a temperature regulating element disposed centrally within the housing and configured to heat and/or cool the therapy surface; a light therapy module comprising a plurality of LEDs disposed circumferentially around the temperature regulating element and directed toward the therapy surface; a plurality of microcurrent electrodes disposed circumferentially around the temperature regulating element and spaced apart from the plurality of LEDs; a control circuit configured to selectively activate the temperature regulating element, the plurality of LEDs, and the plurality of microcurrent electrodes; and a magnetic assembly comprising at least one magnetic element disposed on the housing and configured to magnetically couple the multi-modal therapeutic cap device to a cosmetic container; and wherein the multi-modal therapeutic cap device is configured to be detachably secured to the cosmetic container for storage, and to be detached from the cosmetic container for skin-treatment use.

In one embodiment of the invention, the plurality of LEDs circumferentially surrounds the temperature regulating element, and the plurality of microcurrent electrodes circumferentially surrounds the plurality of LEDs.

In one embodiment of the invention, the temperature regulating element comprises a Peltier module thermally coupled to a metallic heat-transfer plate in contact with the therapy surface.

In one embodiment of the invention, the housing comprises a first end and a second end, the second end including a holder configured to assist a user in holding the multi-modal therapeutic cap device during use.

In one embodiment of the invention, placement of a user's finger on the holder actuates a switch assembly disposed within the second end to initiate operation.

In one embodiment of the invention, the holder comprises a finger ring rotatably coupled to the second end of the housing.

In one embodiment of the invention, the magnetic assembly comprises a plurality of magnetic elements arranged circumferentially around an outer peripheral region of the housing to provide uniform magnetic coupling to the cosmetic container.

In one embodiment of the invention, the therapy surface comprises a light-transmitting cover having electrodes and optical windows aligned with the plurality of LEDs.

According to a second aspect of the present invention, a multi-modal therapeutic cap assembly is provided. The multi-modal therapeutic cap assembly comprises: a connecting cover adapted to be detachably mounted at a mouth of a cosmetic container, the connecting cover defining a storage slot; a multi-modal therapeutic cap device including a housing and a physiotherapy component arranged in the housing, the housing having a working end face, and the physiotherapy component having a functional end exposed at the working end face; wherein the multi-modal therapeutic cap device and the connecting cover are magnetically detachable and attachable between a use state and a storage state; the multi-modal therapeutic cap device having a first magnetic element and the connecting cover having a second magnetic element, the first magnetic element and the second magnetic element are coaxially arranged and magnetically attracted to each other to retain the multi-modal therapeutic cap device within the storage slot; and wherein, in the storage state, a face gap is maintained between the functional end and a bottom of the storage slot to prevent physical contact and surface wear.

In one embodiment of the invention, the connecting cover comprises a covering surface and a receiving surface spaced apart from each other, the covering surface defining a covering groove configured to engage and seal an opening of the cosmetic container, and the receiving surface defining the storage slot configured to receive the multi-modal therapeutic cap device in the storage state.

In one embodiment of the invention, the storage slot includes a stop surface at a notch of the storage slot, and the housing of the multi-modal therapeutic cap device includes a contact surface configured to abut against the stop surface when the multi-modal therapeutic cap device is received within the storage slot.

In one embodiment of the invention, a depth (h) of the storage slot extending inward from the stop surface is greater than a distance (d) between the functional end and the contact surface such that a difference between the depth (h) and the distance (d) defines the face gap, and the face gap has a size between 0.4 mm and 2.0 mm to prevent the functional end from contacting the bottom of the storage slot.

In one embodiment of the invention, a plurality of magnetic elements is disposed circumferentially around an outer peripheral region of the housing and is configured to magnetically couple with corresponding magnetic elements on the connecting cover, thereby providing substantially uniform magnetic attraction around a circumference of the multi-modal therapeutic cap device to secure the multi-modal therapeutic cap device in a storage position.

In one embodiment of the invention, a magnetic element is arranged coaxially with a central electrode and with a temperature regulating element, the magnetic element being disposed behind the temperature regulating element and configured to magnetically couple with a corresponding magnetic element positioned in a storage slot of the connecting cover to retain the multi-modal therapeutic cap device in the storage state.

According to a third aspect of the present invention, a multi-modal therapeutic cap assembly system is provided. The system comprises: a connecting cover having a covering surface and an opposite receiving surface, the covering surface defining a covering groove configured to engage an opening of a container, and the receiving surface defining a storage slot; a first magnetic element disposed adjacent to the receiving surface; and a multi-modal therapeutic cap device removably coupled to the connecting cover, the multi-modal therapeutic cap device comprising: a housing including a first end and a second end, the first end having a connecting surface contacting the receiving surface of the connecting cover, and the second end protruding from the connecting surface and being receivable within the storage slot; a second magnetic element disposed on the first end and magnetically attracted to the first magnetic element; and at least one physiotherapy component disposed within the second end and configured to perform at least one of light therapy, temperature regulation, or microcurrent stimulation at a therapy surface.

In one embodiment of the invention, the housing comprises a bottom shell and a top shell enclosing a space accommodating the at least one physiotherapy component, the second magnetic element being embedded in a groove of the bottom shell.

In one embodiment of the invention, multiple first magnetic elements and multiple second magnetic elements are spaced circumferentially around the receiving groove.

In one embodiment of the invention, the housing further comprises a light-transmitting top cover forming a therapy surface and a heat-conducting cover thermally coupled to a temperature-regulating element.

In one embodiment of the invention, the therapy surface includes at least one first electrode and one second electrode electrically connected to a microcurrent-generating module.

In one embodiment of the invention, the physiotherapy component further includes a circuit board supporting a plurality of LEDs arranged around the temperature-regulating element, the electrodes being spaced apart from the light-therapy lamps.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term “several” means more than one, unless otherwise specified.

In the context of the specification, the term “physical therapy” broadly refers to any energy-based or contact-based skin or tissue treatment, including but not limited to thermal therapy (heating or cooling), microcurrent or electrical stimulation, vibration or massage stimulation, and magnetic stimulation. Accordingly, a “stimulation element” may encompass a Peltier element, resistive heater, cooling module, vibration motor, microcurrent electrode pair, magnetic unit, or any combination thereof, configured to deliver one or more therapeutic modalities either alone or together with light therapy.

In the context of the specification, the term “LED module” refers to one or more light-emitting diode (LED) elements that are electrically connected and configured to emit light of specific wavelengths suitable for therapeutic purposes. The LED module may include drive circuitry, heat dissipation structures, and optical elements such as lenses or diffusers to control light distribution.

Unless otherwise stated, the term “light” as used in this specification encompasses electromagnetic radiation in the visible (380-780 nm) and infrared (780 nm-1000 nm) ranges, particularly red light (620-750 nm) and near-infrared (750-1400 nm) wavelengths commonly used in photobiomodulation therapy. Particular wavelengths which may be selected as the dominant emissive wavelength may include the follow, without any preference to be indicated by order: 400 nm, 405 nm, 420 nm, 430 nm, 450 nm, 465 nm, 515 nm, 530 nm, 532 nm, 590 nm, 630 nm, 633 nm, 640 nm, 650 nm, 655 nm, 660 nm, 670 nm, 680 nm, 780 nm, 785 nm, 810 nm, 830 nm, 840 nm, 850 nm, 860 nm, 870 nm, 904 nm, 915 nm, 980 nm, 1015 nm, 1060 nm, 1065 nm, 1070 nm, 1200, and 1400 nm. As used herein, the term “light therapy” refers to the use of one or more light sources of any type that emit light with a wavelength between about 400 and 1400 nm. The device may also emit blue or ultraviolet light for surface-level treatments such as acne reduction or microbial control.

The red light (approximately 630-660 nm) penetrates deeply into the scalp to stimulate blood circulation and enhance hair follicle activity, thus promoting hair growth and repair. Blue light (around 415-470 nm) exhibits antibacterial properties and is effective in treating scalp acne and reducing inflammation. Green light (approximately 520-540 nm) can help reduce pigmentation and soothe sensitive or irritated scalp tissue. Yellow light (around 580-600 nm) improves oxygen exchange in the cells and aids in detoxifying the scalp, while near-infrared light (800-850 nm) reaches deeper layers to accelerate healing and reduce pain.

In the context of this specification, terms like “light”, “radiation”, “irradiation”, “emission”, and “illumination”, etc., refer to electromagnetic radiation in frequency ranges varying from the visible frequencies to Infrared (IR) frequencies and wavelengths, wherein the range is inclusive of visible light, and IR frequencies and wavelengths. Preferably, it refers to low-level electromagnetic radiation of low-level red and near-infrared (NIR) light. It is to be noted here that IR radiation can be categorized into several categories according to respective wavelength ranges, which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm), and Far IR (15-1000 μm). In this regard, light application is at relatively low energy densities, typically below about 500 mW, as compared to other forms of laser therapy that are used for ablation, cutting, and thermally coagulating tissue. In some instances, electromagnetic radiation can also be in wavelengths in the blue or ultraviolet regions, especially for the treatment of conditions that occur at the skin surface, such as psoriasis or infection.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, Super luminous diodes (“SLD”), or Organic light-emitting diodes (OLED).

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety by reference.

In the context of the specification, the term “stimulation element” refers to may include, but is not limited to, phototherapy, micro-current, magneto therapy, cooling, heating, vibration, electrical pulses, or other forms of therapeutic output.

In the context of the specification, the term “electrotherapy,” “electrical stimulation,” or “microcurrent therapy” refers to the application of therapeutic current to the human body, regardless of the precise frequency, waveform, or current intensity.

In the context of the specification, the term “electrotherapy” may refer to different types of electrotherapy, including but not limited to galvanic, electrotherapy, iontophoresis, microcurrent, and EMS (Electrical Muscle Stimulation), TENS (Transcutaneous Electrical Nerve Stimulation).

In the case of a stimulation element being an electrode, the stimulation element may be embodied as an open-ended conductor. The electrode may then be able to provide Transcutaneous Electrical Nerve Stimulation (TENS), Electronic Muscle Stimulation (EMS), and Microcurrent Electrical Therapy (MET) to the target surfaces. TENS therapy uses low-voltage currents to provide pain relief. Electrical impulses are delivered through electrodes placed on the surface of the body of the user.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelengths, wherein the range is inclusive of visible light, UV and IR frequencies and wavelengths. It is to be noted here that UV radiation can be categorized in several manners depending on respective wavelength ranges, all of which are envisaged to be under the scope of this invention. For example, UV radiation can be categorized as Hydrogen Lyman-α (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm). Similarly, IR radiation may also be categorized into several categories according to respective wavelength ranges, which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm), and Far IR (15-1000 μm).

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1 illustrates a perspective view of a multi-modal therapeutic cap assembly, in accordance with an embodiment of the present invention.

FIG. 2 illustrates a perspective view of a multi-modal therapeutic device and a connecting cap configured to connect to a liquid storage container, in accordance with an embodiment of the present invention.

FIG. 3 illustrates an exploded view of the multi-modal therapeutic cap device, in accordance with an embodiment of the present invention.

FIG. 4 illustrates a cross-sectional view of the multi-modal therapeutic cap assembly of FIG. 2, in accordance with an embodiment of the present invention.

FIG. 5 shows an enlarged view of point B of FIG. 4, in accordance with an embodiment of the present invention.

FIG. 6 shows a front view of the multi-modal therapeutic cap device, in accordance with an embodiment of the present invention.

FIG. 7 illustrates an exploded view of the top perspective view of the multi-modal therapeutic cap assembly, in accordance with an embodiment of the present invention.

FIG. 8 is an exploded view of the multi-modal therapeutic cap device showing another configuration/arrangement of various components, in accordance with an embodiment of the present invention

FIG. 9 illustrates a cross-sectional view of the multi-modal therapeutic cap assembly of FIG. 8, in accordance with an embodiment of the present invention.

FIG. 10 illustrates an exploded view of a multi-modal therapeutic cap device showing physiotherapy components arranged at the inverted end, in accordance with an embodiment of the present invention.

FIG. 11 illustrates a perspective view of a multi-modal therapeutic cap device showing physiotherapy components arranged at the inverted end, in accordance with an embodiment of the present invention.

FIG. 12 illustrates an exploded view of the multi-modal therapeutic cap device showing the arrangement of internal components of FIG. 10, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

Throughout this disclosure, the terms “multi-modal therapeutic cap assembly,” “multifunctional therapy device,” and “therapeutic cap” may be used interchangeably to refer to a cap-shaped device or assembly that integrates one or more stimulation elements and at least one light-therapy module under control of a common circuit.

The present invention provides a multi-modal therapeutic cap assembly/multi-functional therapy device designed for use in conjunction with a cosmetic container or as an independent skincare treatment apparatus. The multi-modal therapeutic cap assembly integrates multiple therapeutic modalities, including light therapy, thermal regulation, microcurrent stimulation, vibration, and magneto-therapy, within a compact housing. The multi-modal therapeutic cap assembly comprises a therapy surface intended for direct contact with the skin, a housing enclosing the electronic control and power modules, and a connecting cover that enables detachable or magnetic coupling with the multi-modal therapeutic cap assembly. The internal system includes a circuit board, power supply, and various therapeutic components that operate independently or in combination to deliver targeted skincare treatments. Through intelligent design and multi-mode operation, the multi-modal therapeutic cap device enhances skincare efficiency, user comfort, and portability, providing a professional-level treatment experience in a single compact unit.

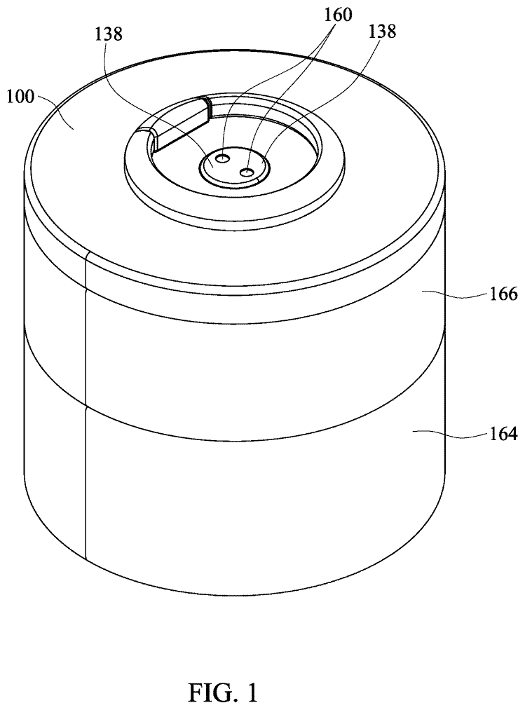

Referring to FIG. 1, in an embodiment, a cosmetic container is provided which consists of a container body 164, a connecting cover 166, and a multi-functional therapy device or multi-modal therapeutic cap device 100. The container body 164 serves as the main container for storing cosmetic liquid or medicament and is configured with an opening at its upper end for attachment with the connecting cover 166. The connecting cover 166 is detachably mounted on the container body 164 and incorporates a connecting ring designed to engage with the upper portion of the container body 164 through a threaded or snap-fit structure, ensuring a stable, leak-proof, and sealed connection. The outer wall of the connecting cover 166 provides a supportive interface for integration with the multi-modal therapeutic cap device 100, allowing the multi-modal therapeutic cap device 100 to be removably secured on top of the container body 164.

In an embodiment, the multi-modal therapeutic cap device 100 is coupled to the connecting cover 166 such that one of the physiotherapy components is configured to connect with the connecting cover 166, enabling the functional modules of the multi-modal therapeutic cap device 100 to perform one or more therapies when it is removed from the connecting cover 166. The arrangement allows users to conveniently store and carry both cosmetic and therapy components in a single integrated unit. The connecting cover 166 is detachably fitted over the top of the multi-modal therapeutic cap device 100 to protect the functional end and its associated functional modules, such as micro-current electrodes or heating/cooling surfaces, from contamination or mechanical damage.

The connecting cover 166 serves as the intermediate structure connecting the container body 164 and the multi-modal therapeutic cap device 100. The connecting cover 166 has two opposite surfaces, a covering surface that faces downward and is configured to seal the container body 164, and an opposite receiving surface designed to accommodate the multi-modal therapeutic cap device 100. The covering surface includes a covering groove that securely engages the neck of the container body 164, thereby closing the container opening and preventing leakage of the skincare product. The receiving surface, on the other hand, includes a receiving slot into which the lower portion of the multi-modal therapeutic cap device 100 is inserted and magnetically retained. This double-surface configuration allows the connecting cover 166 to serve both as a closure cap and as a holder for the therapy device when it is not being used.

The connecting cover 166 ensures mechanical stability and alignment between the container body 164 and the multi-modal therapeutic cap device 100, while simultaneously enhancing hygiene and safety by enclosing the upper end. Such integration allows the user to conveniently use the therapeutic device in conjunction with the cosmetic formulation stored in the bottle, facilitating seamless skincare treatment, storage, and portability.

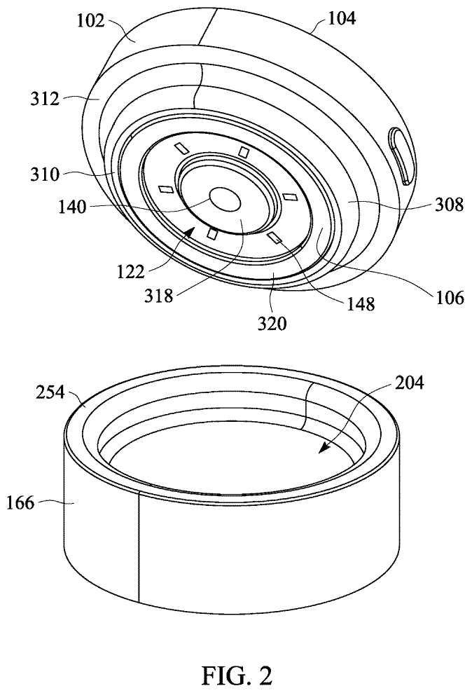

Referring to FIG. 2, the connecting cover 166 is adapted for detachable installation at the mouth of a cosmetic container and is provided with a storage slot 204. The storage slot 204 opens on the side of the connecting cover 166 facing away from the mouth of the cosmetic container. The shape and size of the storage slot 204 correspond to those of the housing 102 of the multi-modal therapeutic cap device 100, ensuring that the multi-modal therapeutic cap device 100 can be smoothly inserted and stably stored. The connecting cover 166 may be attached to the mouth of the cosmetic container through various detachable means such as threaded engagement, snap-fit connection, or magnetic coupling. For instance, when the cosmetic container mouth has an external thread, the inner surface of the connecting cover 166 may be provided with a corresponding internal thread to enable assembly or disassembly by rotation, allowing users to conveniently open the container and access the cosmetic product. It should be understood that a sealing ring may be provided on the inner side of the connecting cover 166 to ensure airtight sealing of the container.

Referring to FIGS. 2, 3, and 4, the multi-modal therapeutic cap device 100 comprises a housing 102 and a physiotherapy component 316 arranged within the housing 102. The shell 102 has a working end face 310, on which a second end 106 of the physiotherapy component 316 is exposed. The working end face 310 may be flat or have a slightly curved contour that conforms to facial anatomy, thereby improving skin contact during therapy. The shape of the second end 106 may vary depending on the specific physiotherapy function performed. The housing 102 may also be provided with control switches or buttons for activating or deactivating the physiotherapy component 316. The device may receive power either via a power cord connected to mains or through a rechargeable battery 158 disposed within the housing 102, preferably a rechargeable battery, with a charging interface provided on the housing 102.

The multi-modal therapeutic cap device 100 and the connecting cover 166 are detachably connected, allowing the device to assume a storage state in which the second end 106 is accommodated within the storage slot 204. In this state, an end face gap 332 is maintained between the bottom of the storage slot 204 and the second end 106.

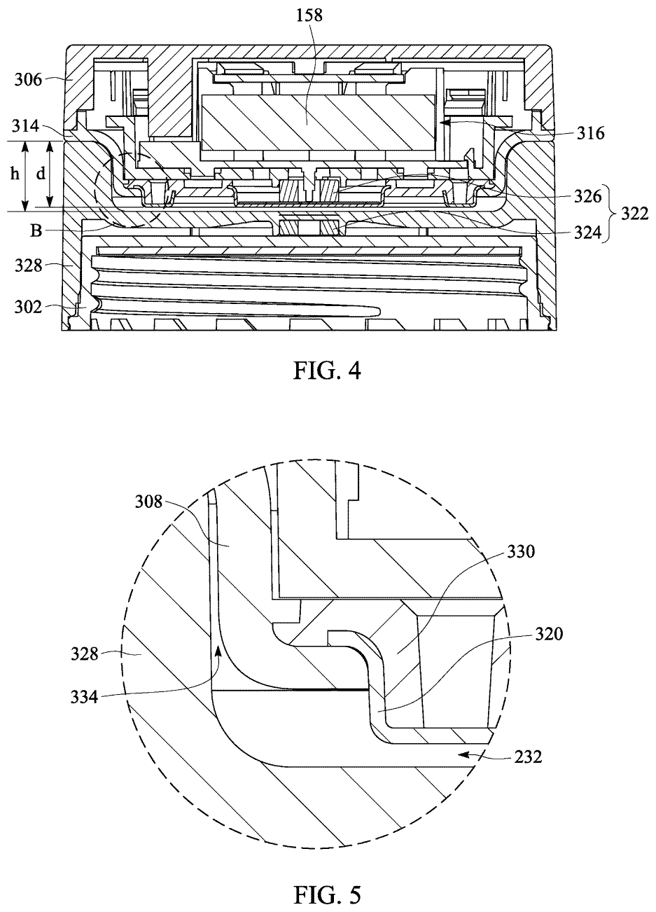

Referring to FIGS. 4 and 5, since the multi-modal therapeutic cap device 100 and the connecting cover 166 are detachably connected, the multi-modal therapeutic cap device 100 can be inserted into the storage slot 204 of the connecting cover 166 by means of a buckle fit, magnetic attraction, or threaded engagement, thereby achieving the storage state. In this position, the second end 106 is received within the storage slot 204, while the end face gap 332 remains between the bottom of the slot and the second end 106. The end face gap 332 effectively prevents direct contact and compression between the second end 106 and the slot bottom, thereby avoiding mechanical damage. Additionally, when the connecting cover 166 is mounted on the cosmetic container and carried by the user, the second end 106 is enclosed within the storage slot 204, preventing it from rubbing against objects such as keys or mobile phones and protecting it from scratches, dust, and debris. This arrangement preserves the integrity of the second end 106.

When physiotherapy is desired, the user may remove the multi-modal therapeutic cap device 100 from the storage slot 204, open the cosmetic container to apply the cosmetic product to the skin, and then activate the physiotherapy component 316. The working end face 310 of the housing 102 is brought into contact with the skin, allowing the second end 106 to act directly on the skin surface. Because the second end 106 is protected from scratches and deformation, it will not cause discomfort such as stinging or redness, even when pressure is applied during use. The intact surface ensures stable and uniform energy output, thereby improving therapeutic performance and enhancing user safety and satisfaction.

Referring to FIG. 2, the notch of the storage slot 204 is provided with a stop surface 254, while the housing 102 includes a contact surface 312 configured to abut the stop surface 254. The depth h of the storage slot 204, measured from the stop surface 254 to the groove bottom, is greater than the distance d between the second end 106 and the contact surface 312. The difference between h and d defines the end face gap 332, which is preferably between 0.4 mm and 2.0 mm.

The stop surface 254 is positioned at the notch of the storage slot 204 and may be formed as an annular or planar structure to limit the insertion depth of the multi-modal therapeutic cap device 100. The stop surface 254 may be perpendicular to the groove wall or inclined at an angle not exceeding 15°, ensuring stable positioning. For example, when the storage slot 204 is circular, the stop surface 254 may be arranged circumferentially in a ring shape with a flat finish to ensure proper seating of the contact surface 312.

The contact surface 312 is formed on the housing 102 of the multi-modal therapeutic cap device 100 and cooperates with the stop surface 254 of the storage slot 204. The contact surface 312 is positioned near the working end face 310, oriented perpendicular or parallel to the inclination of the stop surface 254, ensuring a secure fit. For a cylindrical shell 102, the contact surface 312 may extend circumferentially around the shell and have a diameter matching that of the storage slot 204, ensuring accurate contact when inserted.

The depth h of the storage slot 204 refers to the vertical distance from the stop surface 254 to the groove bottom, while the spacing d between the second end 106 and the contact surface 312 refers to the corresponding vertical distance on the multi-modal therapeutic cap device 100. The end face gap 332, equal to the difference between h and d, is set within 0.4-2.0 mm, typically 0.5-1.5 mm, and preferably 0.6 mm. This gap ensures the second end 106 does not contact the slot bottom, preventing wear. The range also accounts for dust particle sizes (generally 10-300 μm, with larger particles up to 0.3 mm), ensuring sufficient clearance to prevent abrasive accumulation while avoiding excessive spacing that might compromise stability or aesthetics.

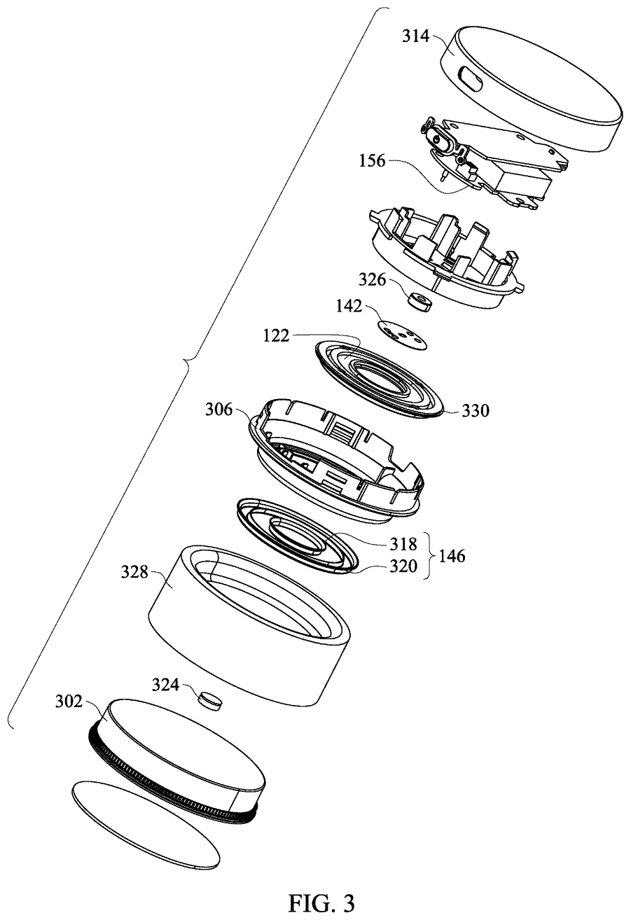

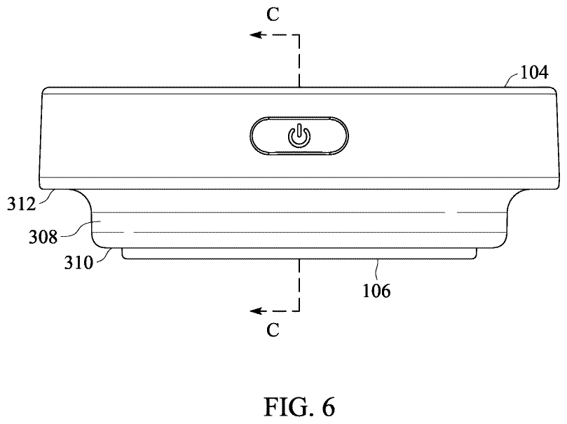

Referring to FIGS. 2, 5, and 6, the housing 102 is provided with a boss 308, on which the working end face 310 is formed. When the multi-modal therapeutic cap device 100 is stored, the boss 308 is inserted into the storage slot 204, and its outer contour matches the inner wall of the groove. The boss 308 may be integrally formed with or fixed to the housing 102. For example, as shown in FIGS. 3 and 4, the housing 102 includes a first shell 306 and a second shell 314, assembled together to form an internal cavity accommodating the physiotherapy component 316. The boss 308 may be provided on the first shell 306. Its shape, cylindrical, square, or oval, is selected to correspond to the shape of the storage slot 204. For a circular slot, the boss 308 is preferably cylindrical and coaxial. The height of the boss 308 ensures proper accommodation of the second end 106 within the slot, maintaining the end face gap 332. The working end face 310, positioned at the end of the boss 308, exposes the second end 106 for direct operation while ensuring precise alignment with the slot.

To store the multi-modal therapeutic cap device 100, the boss 308 is aligned with the storage slot 204 and inserted. As the contours are matched, the boss 308 smoothly slides in until the contact surface 312 abuts the stop surface 254, positioning the device in a stable storage state with the end face gap 332 maintained. The fit between the boss 308 and the storage slot 204 prevents radial movement and avoids friction between the second end 106 and the slot wall during transport.

To enhance safety and ease of insertion, the edges and corners of the storage slot 204 and boss 308, including the notch and bottom edges, may be rounded. This reduces insertion resistance, prevents sharp edges, and enhances user comfort during handling.

Preferably, referring to FIGS. 4 and 5, when the boss 308 is inserted, a side wall gap 334 that gradually widens along the insertion direction is formed between the boss 308 and the groove wall. This taper, typically an outer taper of 0.5-2° for the boss 308 and a corresponding inner taper of 0° or less for the groove wall, facilitates automatic alignment during insertion, reduces resistance, and allows air discharge to avoid a “piston effect.” The widening gap also serves to trap small impurities or droplets, keeping the second end 106 clean during storage.

In some embodiments, the cross-sections of the boss 308 and storage slot 204 are circular and coaxial. The multi-modal therapeutic cap device 100 and connecting cover 166 are detachably connected through a magnetic assembly 322, including a first annular magnet 324 and a second annular magnet 326. The first annular magnet 324 is positioned at the bottom of the groove of the storage slot 204, while the second annular magnet 326 is disposed within the boss 308 or near the second end 106. The two magnets are coaxial and magnetically attract each other to secure the device in the storage state. The magnetic attraction is strong enough to hold the device in place while allowing convenient detachment.

Referring to FIGS. 3 and 4, the connecting cover 166 includes an inner cover 302 and an outer cover 328, with the outer cover 328 surrounding the inner cover 302. The inner cover 302 is detachably connected to the cosmetic container (for instance, by threading), while the storage slot 204 is formed on the outer cover 328. The first annular magnet 324 may be positioned between the inner cover 302 and outer cover 328 within a corresponding groove. In the multi-modal therapeutic cap device 100, the second annular magnet 326 may be positioned on the dorsal side of the electrode or within the boss 308, remaining invisible from the exterior.

Alternatively, the multi-modal therapeutic cap device 100 and the connecting cover 166 may be detachably connected using magnetic attraction, buckle engagement, or threaded coupling. For example, an elastic buckle may be formed on the side wall of the storage slot 204, and a matching ring groove may be provided on the boss 308. During insertion, the buckle deforms to engage the groove, securing the device; during removal, the buckle disengages upon pulling.

Similarly, a threaded connection may be used, wherein the inner wall of the storage slot 204 carries an internal thread, and the outer wall of the boss 308 carries a corresponding external thread, allowing rotational engagement and release.

The physiotherapy component 316 comprises a circuit board 156 and one or more functional modules. The functional module may include at least one of a phototherapy module, a microcurrent stimulation module, a hot compress module, or a vibration module.

Each functional module is electrically connected to the circuit board 156, which supplies power, manages mode switching, and controls operational parameters of the modules. One end of the circuit board 156 is connected to a power supply, and the other end is electrically connected to each functional module through wires or soldering. The circuit board 156 receives user operation commands, such as button inputs on the housing 102, and outputs corresponding control signals to adjust working parameters such as phototherapy intensity, microcurrent level, heating temperature, or vibration frequency.

The phototherapy module includes a light therapy module 148 and a light-transmitting cover 122. The light therapy module 148 is arranged within the housing 102 and electrically connected to the circuit board 156, while the light-transmitting cover 122 is positioned on the working end face 3110. The light therapy module 148 may comprise LED lamp beads, such as red, blue, or near-infrared LEDs, arranged singly or in multiples depending on treatment requirements and distributed in alignment with the light-transmitting cover 122. The light-transmitting cover 122 is a transparent or translucent member fixed to the working end face 310, protecting the light therapy module 148 while allowing uniform light penetration to the facial skin. The wavelength ranges may include red light (620-660 nm), blue light (450-480 nm), and near-infrared light (800-980 nm).

The microcurrent stimulation module comprises at least two microcurrent electrodes 146, which are exposed on the working end face 310 and electrically connected to the circuit board 156. The micro-current electrodes 146 are spaced apart to prevent short circuits and can be connected via wires or conductive columns. The circuit board 156 regulates the intensity and frequency of the microcurrent transmitted through the micro-current electrodes 146 to stimulate facial skin cells.

The hot compress module includes a temperature-regulating element 142 and a thermal conductive member. The temperature-regulating element 142 is arranged within the housing 102, electrically connected to the circuit board 156, and thermally coupled to the thermal conductive member, which is exposed on the working end face 310. The temperature-regulating element 142 may be an NTC sheet, PI heating film, or resistance heating sheet, with temperature control typically ranging between 38° C. and 45° C. The thermal conductive member ensures even heat transfer to the skin and prevents localized overheating.

The vibration module is fixed within the housing 102 and electrically connected to the circuit board 156. It may include a miniature vibration motor attached by screws, snaps, or adhesive. The circuit board 156 controls the vibration frequency and amplitude, and the vibrations are transmitted through the housing 102 and working end face 310 to provide a massage and enhance the absorption of skincare products.

During use, the user selects a desired therapy mode through an operation button connected to the circuit board 156, such as phototherapy alone or a combined mode (e.g., hot compress plus vibration). Upon receiving the command, the circuit board 156 powers and activates the corresponding modules. Each module may operate independently or in combination to provide customized treatment effects.

The functional modules of the physiotherapy component 316 can be combined in various ways depending on the desired application. Single-module configurations include phototherapy, microcurrent stimulation, hot compress, or vibration only. Dual-module configurations may include combinations such as phototherapy+microcurrent, phototherapy+hot compress, phototherapy+vibration, microcurrent+hot compress, microcurrent+vibration, or hot compress+vibration. Triple-module configurations may include phototherapy+microcurrent+hot compress, phototherapy+microcurrent+vibration, phototherapy+hot compress+vibration, or microcurrent+hot compress+vibration. A four-module configuration combines phototherapy, microcurrent stimulation, hot compress, and vibration.

The micro-current electrodes 146, thermal conductive member, and light-transmitting cover 122 collectively define the second end 106.

When both the microcurrent stimulation module and the hot compress module are included, the thermal conductive member may be formed by at least one micro-current electrode 146. The micro-current electrodes 146 can thus serve both electrical and thermal functions, and may be made of medical-grade stainless steel, gold-plated copper, or graphene. The micro-current electrodes 146 may be sheet-shaped, dot-shaped, or ring-shaped, and are exposed on the working end face 310. It is thermally coupled with the temperature-regulating element 142 and electrically connected to the circuit board 156, thereby performing dual microcurrent and heat conduction functions.

At least one micro-current electrode 146 may serve as a central electrode 318, while at least one other electrode serves as a surrounding electrode 320 positioned around the central electrode 318. The light-transmitting cover 122 is arranged in the annular region between the surrounding electrode 320 and the central electrode 318. The central electrode 318, which may be circular or polygonal, provides both electrical and thermal conductivity and serves as a central reference point for the working end face 310. The surrounding electrode 320 may be a single ring or multiple discrete electrodes arranged circumferentially around the central electrode 318, with a fixed spacing to avoid short circuits. Both electrodes are connected to the circuit board 156 to form a microcurrent loop and can also participate in heat transfer.

The surrounding electrode 320 and the central electrode 318 are fixed to the housing 102 by a fixing bracket 330, which includes an opening or light-transmitting cover 122 corresponding to the space between the electrodes. The light therapy module 148 is aligned with the light-transmitting cover 122 to ensure even illumination of the treatment area. This arrangement prevents the electrodes from obstructing light output, thereby achieving overlapping phototherapy and microcurrent stimulation areas for synergistic treatment effects.

The temperature-regulating element 142 of the hot compress module is positioned behind the central electrode 318 and thermally coupled thereto for efficient heat transfer. The second annular magnet 326 of the magnetic assembly 322 is arranged behind the temperature-regulating element 142, coaxially aligned with the temperature-regulating element 142 and the central electrode 318, ensuring optimal use of internal space within the housing 102. The temperature-regulating element 142, central electrode 318, and surrounding electrode 320 are each electrically connected to the circuit board 156 through independent conductive columns to maintain functional independence. The conductive column connected to the central electrode 318 passes through a central opening in the second annular magnet 326, and the temperature-regulating element 142 includes a clearance hole to accommodate this structure, preventing interference between components and ensuring proper assembly and operation.

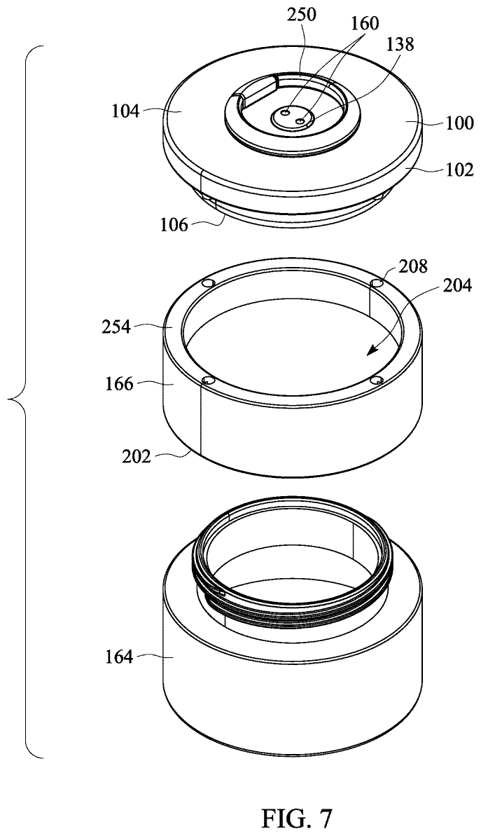

FIG. 7 illustrates an exploded view of the top perspective view of the multi-modal therapeutic cap assembly, in accordance with an embodiment of the present invention. Referring to FIG. 7, in an embodiment of the present invention, another configuration of the cosmetic container is provided. The cosmetic container primarily comprises a multi-modal therapeutic cap device 100, a connecting cover 166, and a container body 164. The overall configuration integrates the multi-modal therapeutic cap device 100 with the connecting cover 166, which can be removably mounted on the mouth of the container body 164. The multi-modal therapeutic cap device 100 functions as both a lid for the container and a skincare treatment instrument, thus improving portability, user convenience, and maintaining hygiene by preventing external contamination of the therapy surface when not in use.

The connecting cover 166 serves as the intermediate structure connecting the container body 164 and the multi-modal therapeutic cap device 100. The connecting cover 166 has two opposite surfaces, a covering surface 200 that faces downward and is configured to seal the container body 164, and an opposite receiving surface designed to accommodate the multi-modal therapeutic cap device 100. The covering surface 200 includes a covering groove 202 that securely engages the neck of the container body 164, thereby closing the container opening and preventing leakage of the skincare product. The receiving surface, on the other hand, includes a storage slot 204 into which the lower portion of the multi-modal therapeutic cap device 100 is inserted and magnetically retained. This double-surface configuration allows the connecting cover 166 to serve both as a closure cap and as a holder for the therapy device when it is not being used.

The multi-modal therapeutic cap device 100 is detachably mounted on the upper side of the connecting cover 166 through a magnetic coupling mechanism. The multi-modal therapeutic cap device 100 includes an internal housing that contains therapeutic modules such as phototherapy lamps, a temperature regulator, and a microcurrent generating module. The second end 106 of the multi-modal therapeutic cap device 100 defines a therapy surface configured to contact the user's skin during operation. When the device is magnetically attached to the connecting cover 166, the second end 106 faces inward, fitting securely into the receiving groove of the cover body, thereby preventing dust or accidental contact. During use, the user can easily detach the multi-modal therapeutic cap device 100 from the connecting cover 166, activate it via a switch assembly, and apply it to the desired skin area for treatment.

The container body 164, positioned below the connecting cover 166, is configured to hold cosmetic or therapeutic products such as creams, lotions, medicaments, or serums. Its upper opening is designed to fit into the covering groove of the connecting cover 166, providing an airtight seal. This container body 164 not only serves as a storage vessel but also forms part of the integrated cosmetic container, wherein the multi-modal therapeutic cap device 100 complements the container's function by enhancing the absorption and efficacy of the stored skincare product through thermal, light, or microcurrent stimulation.

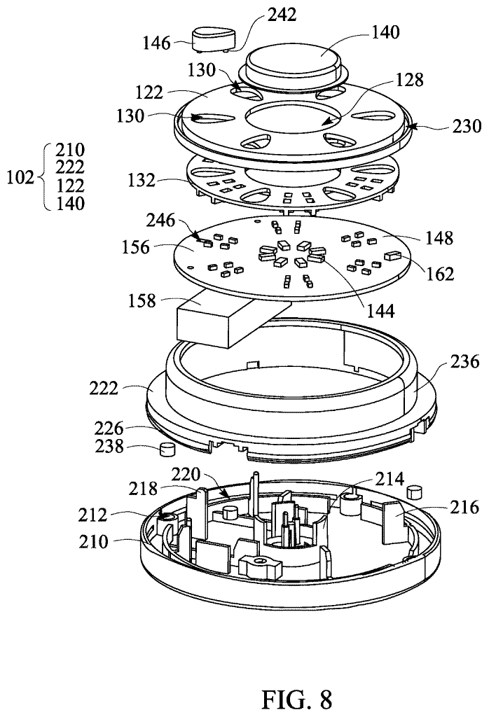

In an embodiment, FIG. 7 and FIG. 8 illustrates the overall assembly relationship of the multi-modal therapeutic cap device 100, showing the arrangement and interaction between a finger ring 250, a switch assembly 138, including a switch assembly 138, and a pair of charging electrodes 160, the multi-modal therapeutic cap device 100, the magnetic connection with the connecting cover 166, and the container body 164. The multi-modal therapeutic cap device 100 is magnetically coupled to the connecting cover 166, forming a combined lid structure capable of performing phototherapy and skincare treatment while also serving as the closure for the container body 164. The connecting cover 166 comprises a covering surface 200 designed to close the container body 164 and an opposite receiving surface for holding the multi-modal therapeutic cap device 100. The receiving surface includes a storage slot 204 configured to accommodate the second end 106 of the multi-modal therapeutic cap device 100, ensuring a flush alignment and protective housing when the device is not in use.

The multi-modal therapeutic cap device 100, positioned above the connecting cover 166, includes an outer housing that encloses various internal therapeutic and electronic components. The multi-modal therapeutic cap device 100 connects magnetically to the connecting cover 166 through a first magnetic element 208 embedded within the connecting cover 166 and a second magnetic element 238 located on the second end 106 of the multi-modal therapeutic cap device 100. This arrangement enables firm attachment while permitting easy detachment for use. The second end 106 of the multi-modal therapeutic cap device 100 defines a connecting surface that aligns with the receiving surface of the connecting cover 166, while the second section of the multi-modal therapeutic cap device 100 extends downward into the storage recess of the connecting cover 166. The second end 106 of the multi-modal therapeutic cap device 100 exposes a therapeutic surface configured to contact the user's skin during operation.

Positioned on the side of the multi-modal therapeutic cap device 100, the switch assembly 138 serves as the main operational interface for activating the phototherapy and temperature-control functions. The switch assembly 138 includes the switch assembly 138, movably mounted to the housing base, and two charging electrodes 160 that extend through corresponding apertures in the switch assembly 138. When the user presses the switch assembly 138, the charging electrodes 160 engage the circuit board housed within the multi-modal therapeutic cap device 100, generating a start signal to initiate the therapeutic modules, such as the phototherapy lamps, temperature regulator module, and microcurrent module. In addition to control functionality, the switch assembly 138 serves as a contact surface for charging, allowing the device's internal battery to be recharged through external connection points. The dual functionality of the switch assembly thus simplifies operation and charging within a single, sealed interface.

The finger ring 250 is attached to the outer surface at a first end 104 of the multi-modal therapeutic cap device 100. The finger ring 250 allows the user to insert a finger through the ring to hold and manoeuvre the multi-modal therapeutic cap device 100 easily during use. When rotated to rest flush against the connecting cover 166, the ring frames the switch assembly at its center, protecting the switch assembly 138 and the charging electrodes 160 from accidental activation. The integration of the finger ring 250 provides both functional convenience and compact portability, ensuring the user can operate the multi-modal therapeutic cap device 100 with precision during application of skincare treatments.

The container body 164 forms the base component of the cosmetic container, which holds cosmetic or therapeutic creams, serums, medicaments, or lotions. The container body's mouth fits securely into the covering groove formed in the upper surface of the connecting cover 166, allowing the multi-modal therapeutic cap device 100 to serve as both a closure cap and a detachable treatment unit. When assembled, the magnetic connection between the connecting cover 166 and the multi-modal therapeutic cap device 100 keeps the device fixed in place over the container body 164, protecting the therapy surface from contamination. In use, the user can easily detach the multi-modal therapeutic cap device 100 from the connecting cover 166, power it on using the switch assembly 138, and hold it using the finger ring 250 to perform light, thermal, or microcurrent therapy after applying skincare products from the container body 164.

In some embodiments, the multi-modal therapeutic cap device further includes a holder portion to facilitate ergonomic handling by the user. The holder may be provided on the first end 104 of the housing 102 and may take the form of a finger ring or loop through which the user can insert one or more fingers during treatment. The finger ring may be rotatably coupled to the first end 104 of the housing so that the device can be conveniently oriented relative to the user's hand while maintaining a stable grip. Alternatively, the holder may be fixedly formed as an annular projection integrally molded with the housing.

In one embodiment, placement of a user's finger on the holder or an outer surface of the bottom shell actuates the switch assembly 138 disposed within the housing. The switch assembly 138 may include a capacitive touch sensor, a pressure-sensitive switch, or a mechanical push button, configured to initiate or terminate operation of one or more physiotherapy functions such as heating, light emission, or microcurrent stimulation. This configuration allows for intuitive activation of the device without separate manual buttons.

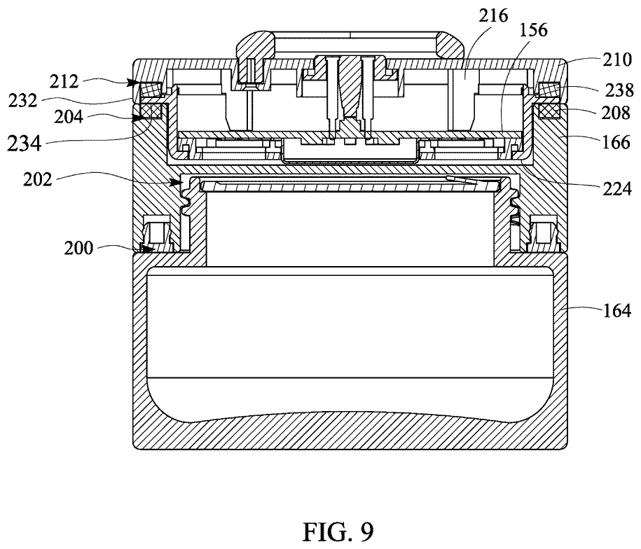

In an embodiment, FIG. 8 and FIG. 9 illustrates the interaction of the layers of the housing 102, which consists of the bottom case 210, the middle case 222, the light-transmitting cover 122, the first section 232 of the housing, the magnets, the circuit board 156, and the rechargeable battery 158, and the structural features that ensure reliable assembly and safe operation.

The first section 232 of the housing is the annular outer portion of the multi-modal therapeutic cap device 100 that surrounds the protruding second section 236. The first section 232 is a part that contacts and rests upon the connecting cover 166 receiving surface when the device is docked. A connecting surface 234 is the lower face of the first section 232, and it is sized and shaped to mate closely with the connecting cover 166 receiving surface and, where provided, with a storage slot 204. The mating at the connecting surface 234 establishes radial and axial location, and it prevents lateral movement of the multi-modal therapeutic cap device 100. At the same time, it is docked and ensures the therapy surface is protected within the storage groove. Because first section 232 surrounds the second section 236, the first section carries the second magnetic element 238 (typically positioned in a second recessed groove 212 of the bottom case or retained between bottom/top shells) so that, when the device is brought to the cover, the second magnetic element on the first section 232 aligns with and magnetically couples to the first magnetic element 208 embedded in the cover body. Thus, the first section 232 and the connecting surface 234 together form the mechanical interface and the primary magnetic attachment region.

In an embodiment, the second section 236 projects downward from the connecting surface 234 and is received into the connecting cover's 166 storage slot 204. The second section ends in the therapy surface or the second end 106, which, in normal use, faces the skin. When docked, the therapy surface or the second end 106 faces the bottom of the storage slot 204 and is therefore shielded from external contamination. The relative clearances between the second section 236 and the walls of the receiving groove are provided and are chosen to permit easy insertion/removal while ensuring the therapy surface is recessed deep enough to be protected. The second section also houses the stimulation elements, such as light therapy module 148, temperature-regulating element 142, micro-current electrodes 146, directly behind or through the light-transmitting cover 122, so that, when undocked, therapies like light, heat, cool, vibration, and microcurrent can be delivered effectively to the skin.

The middle case 222 integrates an inner flanged edge 224, which is an annular inwardly-directed lip formed at one end of the middle case 222. In assembly, the edge of the light-transmitting cover 122 fits within the middle case and bears against the inner flanged edge 224, preventing the top cover from displacing outward and providing a mechanical stop. The inner flanged edge 224 also overlaps the glue groove 230 in the top cover. The glue groove 230 is filled with adhesive during the manufacturing process, so that the inner flanged edge 224 covers the adhesive bead after assembly. This arrangement both secures the top cover and improves water resistance around the top cover perimeter. The inner flanged edge 224 functions as a retention feature, a protective cover for the adhesive joint, and contributes to the overall cleanliness performance of the device.

In an embodiment, the bottom case 210 with its first support ribs 214 and second support ribs 216 creates a lattice that supports and positions the circuit board 156 and the rechargeable battery 158. The circuit board is shown seated on the ribs and constrained axially by contact with the light-transmitting cover 122 and the light-shielding plate 132 between the board and the top cover. Positioning posts 218 engage corresponding positioning holes 246 in the board to provide precise alignment, and the annular groove 220 on the bottom case engages the annular rib 226 on the middle case to lock the two shells together radially. The second recessed groove 212 in the bottom case locates the second magnetic element 238 so that the magnet's pole faces are oriented outward toward the cover body when assembled. These nested ribs, posts, and grooves give the internal modules fixed-toleranced locations so electrical connectors, support legs 242, and thermal interfaces remain aligned to their openings in the light-transmitting cover 122.

In an embodiment, the heat-conducting plate 140 sits inside the first opening 128 of the light-transmitting cover 122 and couples to the temperature-regulating element 142 on the board. The heat-conducting plate 140 provides a direct, low-resistance thermal path from the regulator to the therapy surface; this is necessary for effective heating or cooling of the skin. The light therapy module 148 is arranged around the regulator and is positioned so its emitted light passes through the light-transmitting area of the light-transmitting cover 122; the light-shielding plate 132 provides the aperture/clearance necessary for the lamps and simultaneously conceals the rest of the circuitry from view. The micro-current electrodes 146 are provided with support legs 242, which pass through the second openings 130 of the light-transmitting cover 122 and are electrically connected to the microcurrent generating element 162 on the circuit board 156. This arrangement clearly shows the electrode protrusion or flushness relative to the therapy surface and how that geometry is protected when the second section 236 is seated in the receiving groove.

In an embodiment, the therapy surface or the second end 106 and electrodes are recessed when docked because the second section 236 sits inside the storage slot 204, preventing contamination or accidental contact. The first section 232 with its connecting surface 234 provides a stable annular seating that, together with the magnets in the second recessed groove 212 and the cover's magnetic elements, secures the device concentrically with a predictable pull-in force. The inner flanged edge 224, glue groove 230, and mounted thermal cover 140 produce a sealed region that both transmits desired thermal/optical energy and prevents ingress of contaminants or liquid into the internal electronics.

In another embodiment, a central magnetic element is arranged coaxially with the temperature-regulating element and/or central electrode of the physiotherapy module. The coaxial arrangement provides a balanced magnetic field along the longitudinal axis of the device and enhances axial retention of the therapeutic cap within the connecting cover during storage, while maintaining electrical and thermal symmetry in the active treatment zone.

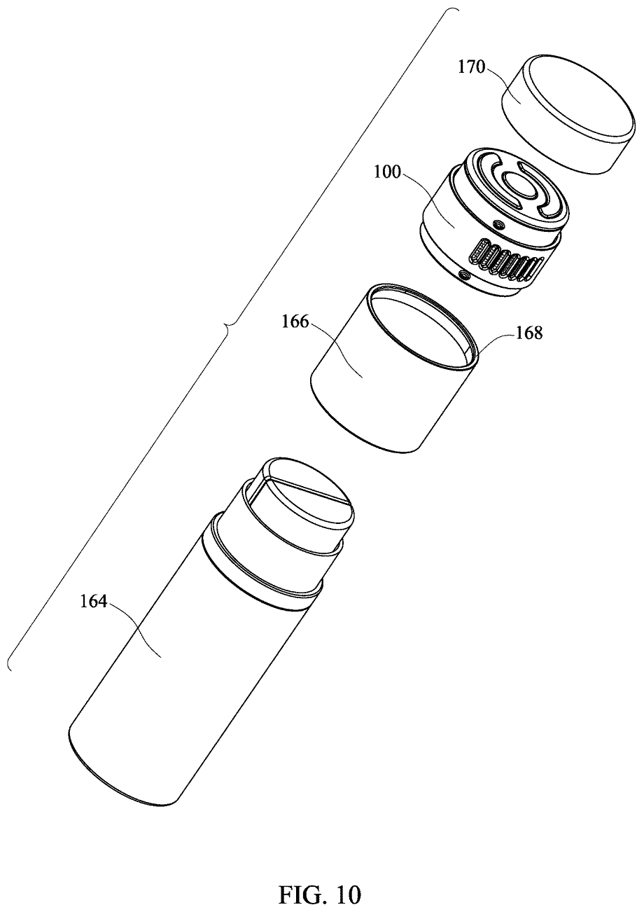

FIG. 10 illustrates an exploded view of a multi-modal therapeutic cap assembly showing physiotherapy components arranged at the inverted end, in accordance with an embodiment of the present invention. Referring to FIG. 10, in an embodiment, an exploded view of the multi-modal therapeutic cap assembly is illustrated, showing the arrangement and connection relationship between a cap 170, multi-modal therapeutic cap device 100, a connecting ring 168, connecting cover 166, and container body 164, demonstrating how the individual components are assembled to form an integrated cosmetic system capable of both skincare product storage and therapeutic treatment. Each part is structurally adapted to fit precisely with adjacent components, thereby ensuring stable mechanical assembly, effective sealing, and convenient disassembly for refilling, cleaning, or maintenance.

The container body 164 constitutes the main reservoir for the cosmetic formulation, such as essence, serum, medicament, or lotion. The container body 164 can be formed as a hollow container, such as bottles, tubes, cases, etc., preferably from transparent or translucent material, enabling the user to visually monitor the remaining content. The upper portion of the container body 164 defines an open mouth configured for coupling with the connecting cover 166. The connecting cover 166 is provided with a covering surface 200 that interfaces with the mouth of the container body 164 either through a threaded connection, snap-fit, or bayonet coupling, forming a secure and leak-proof joint to prevent spillage of the cosmetic fluid during handling or transportation while maintaining an aesthetically unified design with the attached therapeutic device.

The connecting cover 166 is positioned above the container body 164, which acts as an intermediary coupling member between the container body 164 and the multi-modal therapeutic cap device 100. The connecting cover 166 is formed as a cylindrical or tubular member with the receiving surface at the upper end and the covering surface at the lower end, and the edges form the connecting ring 168. The connecting ring 168 fits into the upper end of the connecting cover 166, providing a firm structural interface for mounting the multi-modal therapeutic cap device 100. The connecting ring 168 may be made of a resilient or metallic material to provide an elastic clamping force, thereby ensuring that the multi-modal therapeutic cap device 100 and the container body 164 can be easily attached or detached while maintaining a tight connection during use. This arrangement facilitates quick assembly and replacement of the stimulation device without affecting the integrity of the cosmetic container.

The multi-modal therapeutic cap device 100 detachably connects to the receiving surface of the connecting cover 166 through the connecting ring 168. The lower portion or the second end 106 of the multi-modal therapeutic cap device 100 engages with the connecting ring 168, while its first end 104 remains exposed for skin contact and treatment. This modular configuration allows the device to receive power through internal connections within the housing and operate independently while being physically supported by the bottle assembly. The integration of the multi-modal therapeutic cap device 100 with the container enhances user convenience by combining cosmetic application with light, temperature, or microcurrent therapy in a single, handheld unit. The smooth interface between the connecting cover 166 and the device ensures stable alignment, minimizing vibration and ensuring proper function during therapeutic operation.

Furthermore, the cap 170 is designed to cover the upper portion or first end 104 of the multi-modal therapeutic cap device 100, enclosing the exposed first end 104 of the phototherapy device when not in use. The cap 170 may include an inner sleeve structure that frictionally engages the outer wall of the device or the connecting ring 168, ensuring a snug fit that protects the functional components from dust, moisture, and mechanical damage. When assembled, the cap, stimulation device, connecting ring, connecting cover, and container body form a unified cosmetic container or kit.

In an embodiment, the multi-modal therapeutic cap device 100 further includes the cap 170, which is detachably connected to the first end 104. This allows the first end 104 to be covered, keeping it clean. The first end 104 is provided with a first plug section 116, and the second end 106 is provided with a second plug section 118 for detachably connecting the cap 170 and the connecting cover 166. The outer periphery of the first plug section 116 features a first protrusion 112 that engages with the cap 170, and the second plug section 118 features a second protrusion 114 that engages with the inner wall of the connecting cover 166. The connecting cover 166 has a plate-like structure formed in the inner space of the connecting cover, which serves as the separator between the container body 164 and the multi-modal therapeutic cap device 100, so that the electrical circuitry of the multi-modal therapeutic cap device 100 can be protected from the medicament present in the container body 164. Thus, during use, the connecting cover 166 can also be placed over the second end 106 for easy handling and to help prevent the connecting cover 166 from being lost.

In an embodiment, the first protrusion 112 and the second protrusion 114 can comprise magnetic elements configured to provide magnetic coupling between corresponding magnetic components on the cap 170 and the connecting cover 166, respectively. The magnetic elements embedded within the protrusions (112 and 114) enable a secure yet detachable connection, ensuring proper alignment and positional stability of the multi-modal therapeutic cap device 100 during use and storage. The magnetic attraction between the corresponding magnetic elements facilitates convenient attachment and removal of the cap 170, multi-modal therapeutic cap device 100, and the connecting cover 166 without the need for excessive mechanical force or complex locking structures. This magnetic coupling arrangement not only enhances user convenience but also improves the overall durability of the connection interfaces by reducing wear from repeated engagement and disengagement.

To enhance positional stability and ensure uniform magnetic coupling, the magnetic attachment interface may include a plurality of magnetic elements distributed circumferentially around the periphery of the receiving groove or annular housing. The corresponding magnetic elements on the mating component may be positioned in a complementary arrangement such that the magnetic attraction forces are balanced in the radial direction, preventing tilting or displacement of the therapy device during attachment or operation.

In an embodiment of the present invention, the connecting cover 166 at its first end has a covering surface 200 and a receiving surface at the second end. The covering surface defines a covering groove 202 configured to engage an opening of a container, and the receiving surface defines a storage slot 204.

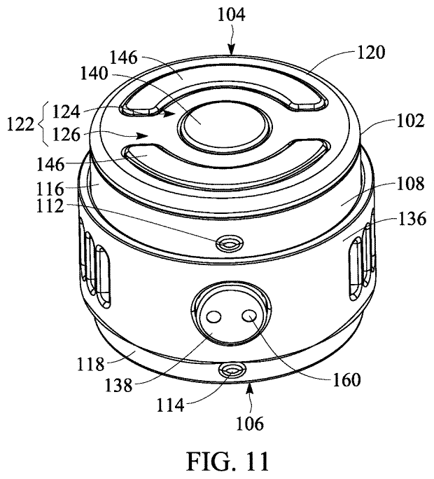

Referring to FIG. 11, a perspective view of a multi-modal therapeutic cap device 100 showing physiotherapy components arranged at the inverted end is illustrated, showing the arrangement and interconnection of its major components. The multi-modal therapeutic cap device 100 includes a housing 102, a first end 104, the second end 106, a barrel 108, an outer shell 136, a heat-conducting plate 140, a micro-current electrodes 146, a light-transmitting cover 122 with a first light therapy area 124 and a second light therapy area 126, and various connecting and protective structures such as a first plug section 116, the second plug section 118, the first protrusion 112, the second protrusion 114, the switch assembly 138, and the charging electrode 160.

The housing 102 forms the main body of the phototherapy device and comprises a barrel 108 surrounded by the outer shell 136. The barrel 108 serves as the structural core for housing internal components such as the circuit board, battery, microcurrent elements, phototherapy elements, and temperature control elements. The outer shell 136 is fitted around the barrel 108 to provide insulation, aesthetic appearance, and protection from external impact. The housing 102 defines two opposing ends, the first end 104, which is the therapeutic interface that comes into contact with the user's skin, and the second end 106, which connects detachably to the container body through a connecting cover. The outer periphery of the housing is provided with the first protrusion 112 and the second protrusion 114 to improve grip and to cooperatively engage with the inner wall of the cap or connecting structure, ensuring mechanical stability during operation or storage.

In certain embodiments, the bottom shell of the housing may be provided with a plurality of inner and outer support ribs or posts that extend upward toward the interior of the device. These ribs may serve as mechanical reinforcement structures for positioning and supporting a circuit board, light-therapy modules, or a light-transmitting top cover, thereby maintaining the relative spacing between the functional components and improving overall assembly stability and heat dissipation.

In an embodiment, the first protrusion 112 and the second protrusion 114 can be provided with or formed as magnetic elements configured to magnetically couple with corresponding magnetic components on the cap 170 and the connecting cover 166, respectively, allowing the multi-modal therapeutic cap device 100 to be securely and accurately positioned while enabling effortless attachment and detachment. The magnetic coupling between the respective elements ensures stable fixation of the cap 170 and the connecting cover 166 without relying solely on mechanical interlocking, thereby enhancing user convenience. Furthermore, the use of magnetic elements minimizes wear from repeated use and maintains the structural integrity and alignment of the assembled components, ensuring a firm yet easily releasable connection during both storage and operation.

At the first end 104 of the device, the heat-conducting plate 140 and the micro-current electrodes 146 are arranged to perform thermal and microcurrent therapy. The heat-conducting plate 140 is positioned at the central region of the first end 104 and may be made of a conductive metal such as stainless steel or aluminium. It can act as both a heat transfer surface for cooling or heating and as an electrode for microcurrent discharge. Surrounding the heat-conducting plate 140 are micro-current electrodes 146 spaced evenly around it, which provide microcurrent therapy during use. These micro-current electrodes 146, together with the heat-conducting plate 140, define distinct phototherapy zones, specifically, the first light therapy area 124 located between the heat-conducting plate 140 and the surrounding micro-current electrodes 146, and the second light therapy area 126 formed between adjacent micro-current electrodes 146. The overall light-transmitting cover 122 ensures that therapeutic light emitted from internal LED modules passes through to the skin for treatment. This arrangement may allow simultaneous or selective application of heat, light, and microcurrent therapies.

The second end 106 of the housing 102 is designed with a first plug section 116 and the second plug section 118, each having a corresponding first protrusion 112 and the second protrusion 114 that help the stimulation device to lock securely with the connecting cover 166 of the container body 164. The first plug section 116 and the second plug section 118 are shaped to allow quick attachment or detachment, facilitating easy replacement or charging. The switch assembly 138 is positioned on the outer surface of the housing and is configured to control internal functional elements such as the temperature control component, LED light source, and microcurrent generator. The charging electrode 160 is provided adjacent to the switch assembly 138 and is designed for magnetic connection to an external charging line, allowing convenient recharging of the built-in battery without requiring disassembly.

In an embodiment, a multi-modal therapeutic cap device 100 capable of performing light therapy, microcurrent stimulation, and thermal regulation is provided. The ergonomic arrangement of the micro-current electrodes 146, the heat-conducting plate 140, and the light-transmitting cover 122 (the first light therapy area 124 and the second light therapy area 126) ensures uniform treatment over the skin surface, while the robust housing 102 with integrated first plug section 116 and the second plug section 118, and the first protrusion 112 and the second protrusion 114 ensures secure mounting to the cosmetic container assembly. The inclusion of the switch assembly 138 and the charging electrode 160 further enhances user convenience, enabling intuitive operation and easy recharging, thereby making the phototherapy device an integral and reusable therapeutic component of the multimodal cosmetic system.

In some embodiments, the electrical connection between the therapy head and the annular housing, or between the therapeutic cap and an external charging base, may be established through spring-loaded pin contacts, also referred to as pogo-pins. The corresponding mating surface may include conductive pads or terminals aligned with the spring-loaded pins to enable reliable electrical engagement when the components are assembled. In other embodiments, inductive coupling elements may be used in place of direct contacts to achieve wireless power transfer and communication between the control circuit and an external power supply or charging station.

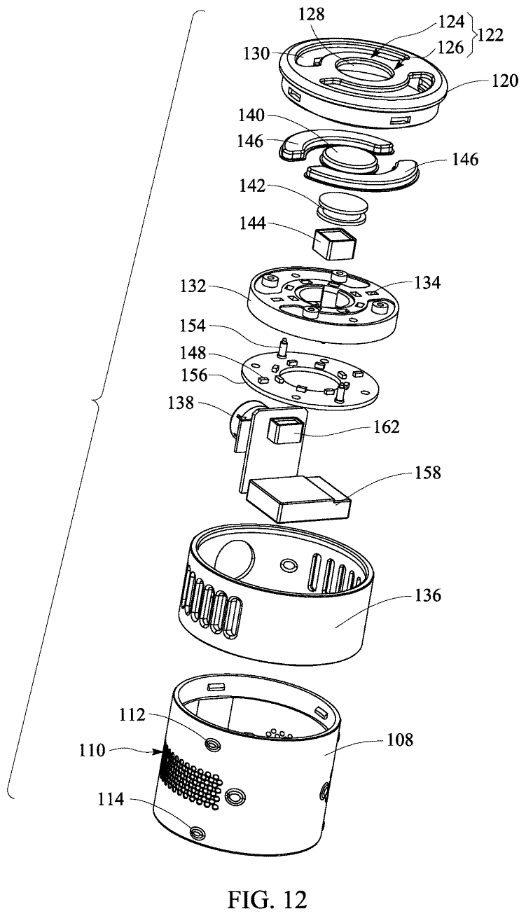

Referring to FIG. 12, in an embodiment, the multi-modal therapeutic cap device 100 exposes the layered internal structure that delivers light therapy, temperature therapy, vibrational therapy, magneto-therapy, ultrasonic wave therapy, and micro-current therapy. The outermost elements include a limit cover 120 and a light-shielding plate 132, which together form the visible first-end. The limit cover 120 is a light-transmitting member that contains a first opening 128 at which the heat-conducting plate 140 is located and a second opening 130 at which the micro-current electrodes 146 are located, thereby allowing the heat-conducting plate 140 and micro-current electrodes 146 to protrude for skin contact while protecting internal components. Surrounding these front elements, the barrel 108 and the outer shell 136 provide the primary structural enclosure, the cylinder holds the internal modules, and the outer shell provides insulation, ergonomic form, and secondary heat-dissipation passages, including a plurality of heat-dissipation holes 110.

Centrally located in the multi-modal therapeutic cap device 100 is the heat-conducting plate 140 with an associated temperature-regulating element 142 and a radiator/heat sink 144. The heat-conducting plate 140 is mounted in a way that its one side is exposed through the first opening 128 and can protrude slightly from the limit cover 120 to make direct thermal contact with the skin; the temperature-regulating element 142 which may be a heating element or cooling element, depending on mode, is fixed to the non-exposed side of the heat-conducting plate 140 and is thermally coupled to the radiator/heat sink 144 to remove or distribute heat. Thermal grease or other conductive interface material may be provided between the temperature control element and the heat-conducting plate to ensure efficient heat transfer. This arrangement ensures that the heat-conducting plate is held rigidly by the limit cover while the radiator sits closer to the device's rear end and vents heat out through the shell holes.

Encircling the heat-conducting plate 140 are the micro-current electrodes 146 and the light-emitting subsystem, which includes a light therapy module 148, a circuit board 156, a plurality of LED lamp beads, and one or more conductive pins 154, which together define the device's phototherapy, thermal, vibrational, magnetic, ultrasonic wave, and microcurrent treatment regions. The light-transmitting cover 122 on the front face is subdivided into the first light therapy area 124, the region between the heat-conducting plate 140 and the surrounding micro-current electrodes 146, and the second light therapy area 126, regions formed between adjacent micro-current electrodes 146. The light therapy module 148 comprises a circuit board 156 populated with multiple LED lamp beads and connected conductive pins/needles 154 that electrically interface to the circuit board 156. The circuit board 156 is nested behind the light-shielding plate 132, so that the LEDs align with light-transmitting holes 134 and the light-transmitting cover 122, thereby delivering targeted optical therapy while the micro-current electrodes 146 remain exposed through the second openings 130 to provide microcurrent therapy.

In operation, when the multi-modal therapeutic cap device 100 is activated, a microcurrent circuit loop is formed through the coordinated interaction of its internal electronic components and the user's skin. The circuit board 156 housed within the multi-modal therapeutic cap device 100 serves as the control and power distribution hub, electrically connected to a microcurrent generating element 162, a power source such as a rechargeable battery 158, and multiple micro-current electrodes 146, along with the heat-conducting plate 140 positioned at the first end 104. During use, when the therapy surface of the device comes into contact with the skin, the heat-conducting plate 140 acts as one pole of the circuit, while one or more of the micro-current electrodes 146 positioned around it function as the opposite pole. The skin itself completes the electrical path between these electrodes, thereby closing the circuit and allowing a controlled microcurrent to flow through the localized tissue. The microcurrent generating element 162 precisely regulates current intensity and waveform to ensure safe, effective stimulation that mimics the body's natural bioelectric signals, promoting cellular activity and enhancing the absorption of skincare formulations. This configuration allows the user to receive consistent and gentle electrical stimulation across the treatment area while maintaining safety through current-limiting circuits and intelligent control integrated into the circuit board 156.

In an embodiment, the multi-modal therapeutic cap device 100 also provides thermal therapy through the integration of a temperature-regulating element 142 coupled to the heat-conducting plate 140. The temperature-regulating element 142 may include a semiconductor thermoelectric module such as a Peltier element, a resistive heating element, or a thermoelectric cooling component, depending on whether heating or cooling therapy is desired. The temperature-regulating element 142 is electrically connected to the circuit board 156, which governs its operation through precise feedback control. When activated, electrical energy from the rechargeable battery 158 is supplied to the temperature-regulating element 142, causing it to either generate or absorb heat. This thermal energy is then efficiently transferred to the heat-conducting plate 140, which is positioned at the first end 104 of the device and directly contacts the user's skin. The heat-conducting plate 140, made of a thermally conductive metal such as aluminium or stainless steel, ensures uniform temperature distribution across the therapy surface. The generated heat or cold penetrates the superficial layers of the skin, stimulating microcirculation, relaxing facial muscles, and improving the absorption of applied cosmetic formulations. To maintain safety and comfort, the circuit board 156 may include temperature sensors or feedback loops that continuously monitor surface temperature and automatically adjust power input to prevent overheating or excessive cooling. This controlled thermal regulation allows the device to deliver consistent and therapeutic temperature effects tailored to user needs, providing both soothing and revitalizing benefits to the skin.

In an embodiment, the multi-modal therapeutic cap device 100 delivers light therapy through a light therapy module 148 comprising the circuit board 156 populated with multiple LED lamp beads positioned behind the light-transmitting cover 122. When the multi-modal therapeutic cap device 100 is powered on, the circuit board 156 activates the LED lamp beads to emit specific wavelengths of light, such as red, blue, or near-infrared, depending on the desired therapeutic effect. The emitted light passes through the light-transmitting cover 122 and reaches the skin surface, where it penetrates different tissue layers to stimulate cellular activity, enhance collagen production, and reduce inflammation. The arrangement of the light therapy module 148, comprising a plurality of LEDs around the heat-conducting plate 140 and the micro-current electrodes 146, ensures uniform illumination, allowing light therapy to be applied simultaneously with thermal and microcurrent treatments for synergistic skincare benefits.

In an embodiment, the multi-modal therapeutic cap device 100 may further provide vibrational therapy through the integration of a micro vibration motor housed within the multi-modal therapeutic cap device 100 and electrically connected to the circuit board. When activated, the vibration motor generates controlled mechanical oscillations that are transmitted through the internal structure to the heat-conducting plate and surrounding therapy surface. These fine vibrations create a gentle massaging effect on the skin, stimulating microcirculation, relaxing facial muscles, and enhancing the absorption of skincare formulations. The vibrational therapy also assists in reducing puffiness and fatigue by promoting lymphatic drainage and improving skin tone. The circuit board regulates the frequency and amplitude of the vibrations, allowing the user to experience a consistent and comfortable massage effect that can be synchronized with other therapeutic functions such as light, thermal, or microcurrent therapy for comprehensive skincare treatment.

In an embodiment, the multi-modal therapeutic cap device 100 may also provide magneto-therapy by incorporating one or more magnetic field-generating elements within the housing or adjacent to the therapy surface. These magnetic elements may be permanent magnets or electromagnetic coils connected to the circuit board 156, which controls the generation and modulation of magnetic fields. When the device is activated, a low-intensity static or pulsed magnetic field is produced and directed toward the user's skin through the heat-conducting plate or surrounding areas. The magnetic field interacts with biological tissues to promote microcirculation, reduce inflammation, and stimulate cellular repair processes. This gentle, non-invasive therapy supports natural healing mechanisms and enhances the absorption of skincare products applied to the treatment area. The integration of magneto-therapy with light, microcurrent, and thermal therapies enables a synergistic rejuvenation effect, offering a holistic and scientifically balanced skincare treatment.

In an embodiment, the circuit board 156 and rechargeable battery 158 are mounted within the barrel 108 and electrically connected to the temperature-regulating element 142, a micro-current generating element 162, and to the circuit board 156. The switch assembly 138 provides user control and carries the charging electrode 160 for magnetic charging. The first protrusion 112 and the second protrusion 114, and the first plug section 116 and the second plug section 118 are peripheral features that cooperate with the connecting cover 166 or bottle-mounting ring to secure the device and prevent unintended rotation or loss of the cap.

The embodiment of the present application also provides a cosmetic apparatus comprising a cosmetic container and the multi-modal therapeutic cap device as described in any of the preceding embodiments, and the cover body 166 of the multi-modal therapeutic cap device is detachably installed at the mouth of the cosmetic container.

Cosmetic containers can be used to hold liquids such as creams, lotions, or facial cleansers, and cosmetic containers have mouths for the liquid inside to flow out or remove the paste. When the cover body 166 is installed at the mouth of the cosmetic container, the liquid outlet is closed by the cover.

Understandably, skin care products such as creams, lotions, and facial cleansers are a type of cosmetic.

The cosmetic apparatus of the embodiment of the present application has the beneficial effect brought about by the physiotherapy cover in any of the above embodiments, and will not be repeated here.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A multi-modal therapeutic cap device for promoting absorption of skincare compositions, comprising:
a housing defining a therapy surface;
a temperature regulating element disposed centrally within the housing and configured to heat and/or cool the therapy surface;
a light therapy module comprising a plurality of LEDs disposed circumferentially around the temperature regulating element and directed toward the therapy surface;
a plurality of microcurrent electrodes disposed circumferentially around the temperature regulating element and spaced apart from the plurality of LEDs;
a control circuit configured to selectively activate the temperature regulating element, the plurality of LEDs, and the plurality of microcurrent electrodes; and
a magnetic assembly comprising at least one magnetic element disposed on the housing and configured to magnetically couple the multi-modal therapeutic cap device to a cosmetic container; and
wherein the multi-modal therapeutic cap device is configured to be detachably secured to the cosmetic container for storage, and to be detached from the cosmetic container for skin-treatment use.

2. The multi-modal therapeutic cap device of claim 1, wherein the plurality of LEDs circumferentially surround the temperature regulating element, and the plurality of microcurrent electrodes circumferentially surround the plurality of LEDs.

3. The multi-modal therapeutic cap device of claim 1, wherein the temperature regulating element comprises a Peltier module thermally coupled to a metallic heat-transfer plate in contact with the therapy surface.

4. The multi-modal therapeutic cap device of claim 1, wherein the housing comprises a first end and a second end, the second end including a holder configured to assist a user in holding the multi-modal therapeutic cap device during use.

5. The multi-modal therapeutic cap device of claim 4, wherein placement of a user's finger on the holder actuates a switch assembly disposed within the second end to initiate operation.

6. The multi-modal therapeutic cap device of claim 4, wherein the holder comprises a finger ring rotatably coupled to the second end of the housing.

7. The multi-modal therapeutic device of claim 1, wherein the magnetic assembly comprises a plurality of magnetic elements arranged circumferentially around an outer peripheral region of the housing to provide uniform magnetic coupling to the cosmetic container.

8. The multi-modal therapeutic device of claim 1, wherein the therapy surface comprises a light-transmitting cover having electrodes and optical windows aligned with the plurality of LEDs.

9. A multi-modal therapeutic cap assembly, comprising:
a connecting cover adapted to be detachably mounted at a mouth of a cosmetic container, the connecting cover defining a storage slot;
a multi-modal therapeutic cap device including a housing and a physiotherapy component arranged in the housing, the housing having a working end face, and the physiotherapy component having a functional end exposed at the working end face;
wherein the multi-modal therapeutic cap device and the connecting cover are magnetically detachable and attachable between a use state and a storage state;
wherein the multi-modal therapeutic cap device having a first magnetic element and the connecting cover having a second magnetic element, the first magnetic element and the second magnetic element are coaxially arranged and magnetically attracted to each other to retain the multi-modal therapeutic cap device within the storage slot; and
wherein, in the storage state, a face gap is maintained between the functional end and a bottom of the storage slot to prevent physical contact and surface wear.

10. The multi-modal therapeutic cap assembly of claim 9, wherein the connecting cover comprises a covering surface and a receiving surface spaced apart from each other, the covering surface defining a covering groove configured to engage and seal an opening of the cosmetic container, and the receiving surface defining the storage slot configured to receive the multi-modal therapeutic cap device in the storage state.

11. The multi-modal therapeutic cap assembly of claim 10, wherein the storage slot includes a stop surface at a notch of the storage slot, and the housing of the multi-modal therapeutic cap device includes a contact surface configured to abut against the stop surface when the multi-modal therapeutic cap device is received within the storage slot.

12. The multi-modal therapeutic cap assembly of claim 11, wherein a depth (h) of the storage slot extending inward from the stop surface is greater than a distance (d) between the functional end and the contact surface such that a difference between the depth (h) and the distance (d) defines the face gap, and the face gap has a size between 0.4 mm and 2.0 mm to prevent the functional end from contacting the bottom of the storage slot.

13. The multi-modal therapeutic cap assembly of claim 9, wherein a plurality of magnetic elements are disposed circumferentially around an outer peripheral region of the housing and are configured to magnetically couple with corresponding magnetic elements on the connecting cover, thereby providing substantially uniform magnetic attraction around a circumference of the multi-modal therapeutic cap device to secure the multi-modal therapeutic cap device in a storage position.

14. The multi-modal therapeutic cap assembly of claim 9, wherein a magnetic element is arranged coaxially with a central electrode and with a temperature regulating element, the magnetic element being disposed behind the temperature regulating element and configured to magnetically couple with a corresponding magnetic element positioned in a storage slot of the connecting cover to retain the multi-modal therapeutic cap device in the storage state.

15. A multi-modal therapeutic cap assembly system, comprising:
a connecting cover having a covering surface and an opposite receiving surface, the covering surface defining a covering groove configured to engage an opening of a container, and the receiving surface defining a storage slot;
a first magnetic element disposed adjacent to the receiving surface; and
a multi-modal therapeutic cap device removably coupled to the connecting cover, the multi-modal therapeutic cap device comprising:
a housing including a first end and a second end, the first end having a connecting surface contacting the receiving surface of the connecting cover, and the second end protruding from the connecting surface and being receivable within the storage slot;
a second magnetic element disposed on the first end and magnetically attracted to the first magnetic element; and
at least one physiotherapy component disposed within the second end and configured to perform at least one of light therapy, temperature regulation, or microcurrent stimulation at a therapy surface.

16. The multi-modal therapeutic cap assembly system of claim 15, wherein the housing comprises a second end and a first end enclosing a space accommodating the at least one physiotherapy component, the second magnetic element being embedded in a groove of the second end.

17. The multi-modal therapeutic cap assembly system of claim 15, wherein multiple first magnetic elements and multiple second magnetic elements are spaced circumferentially around the receiving groove.

18. The multi-modal therapeutic cap assembly system of claim 15, wherein the housing further comprises a light-transmitting top cover forming a therapy surface and a heat-conducting cover thermally coupled to a temperature-regulating element.

19. The multi-modal therapeutic cap assembly system of claim 18, wherein the therapy surface includes at least one first electrode and one second electrode electrically connected to a microcurrent-generating module.

20. The multi-modal therapeutic cap assembly system of claim 19, wherein the physiotherapy component further includes a circuit board supporting a plurality of LEDs arranged around the temperature-regulating element, the electrodes being spaced apart from the light-therapy lamps.
